(19) United States
(12) Patent Application Publication
(10) Pub. No.: US 2015/0320780 A1
(43) Pub. Date: Nov. 12, 2015

(54) MIXTURE OF FUCOSYLATE LACTOSES

(71) Applicant: GLYCOM A/S, Kongens Lyngby (DK)

(72) Inventors: Elise Champion, Toulouse (FR); Gyula Dekany, Sinnamon Park, Queensland (AU); Nikolay Khanzhin, Humlebæk (DK); Pierre Chassagne, Beaumont (FR); Györgyi Osztrovszky, Kisvárda (HU)

(21) Appl. No.: 14/651,710

(22) PCT Filed: Dec. 13, 2013
(86) PCT No.: PCT/DK2013/050431

§ 371 Date: Jun. 12, 2015

(30) Foreign Application Priority Data
Dec. 14, 2012 (DK) PA 2012 70787

(51) Int. Cl. A61K 31/7016; A23L 1/09; A23L 1/29; C12P 19/14; C12P 19/12

(52) U.S. Cl. CPC A61K 31/7016; C12P 19/14; C12P 19/12; A23L 1/296; A23L 1/09; A23V 2002/00

(57) ABSTRACT

A method to form a mixture containing at least one of 2′-FL (2′-O-fucosyllactose, Fuc(α1-2)Gal(β1-4)Glc) and 3-FL (3-O-fucosyllactose, Gal(β1-4)[Fuc(α1-3)]Glc) characterized in that DFL (difucosyl-lactose, Fuc(α1-2)Gal(β1-4)[Fuc(α1-3)]Glc) is subjected to partial hydrolysis, e.g. enzymatic hydrolysis or acid hydrolysis.

### FIELD OF THE INVENTION

The present invention relates to a process for producing a mixture containing at least one of 2′-FL (2′-O-fucosyllactose, Fuc(α1-2)Gal(β1-4)(Glc) and 3-FL (3-O-fucosyllactose, Gal(β1-4)[Fuc(α1-3)]Glc), and optionally containing DFL (difucosyl-lactose, Fuc(α1-2)Gal(β1-4)[Fuc(α1-3)]Glc).

### BACKGROUND OF THE INVENTION

Human milk oligosaccharides (HMOs) have become of great interest in the past few years due to their important functions in human development. To date, the structures of at least 115 HMOs have been determined, and considerably more are probably present in human milk. DFL, 2′-FL and 3-FL (Scheme 1) are considered to be among the more important HMOs.

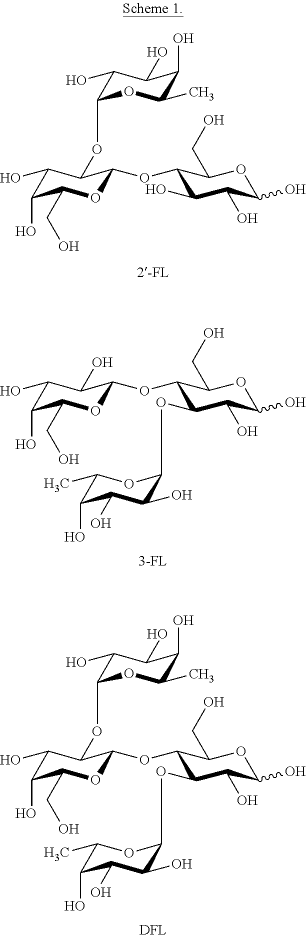

To date, ways of making large volumes of DFL, 2′-FL and 3-FL have not been available. The isolation of fucosylated oligosaccharides from human milk has been rather difficult, even in milligram quantities, due to the presence of a large number of other similar oligosaccharides in human milk. This problem has not been solved by current biotechnology or synthetic chemistry technology.

DFL, 2′-FL and 3-FL are considered to be among the more important HMOs because of their nutritional value (see WO 2012/158517). Because of the growing commercial interest in nutritional formulations and supplements containing HMOs, there has been a need for a low cost method of making such HMOs and mixtures thereof.

DFL was first isolated from mother's milk and its structure was elucidated with standard chemical methods including acid hydrolysis (Kuhn et al, Liebigs Ann. Chem. 611, 249 (1958)).

### SUMMARY OF THE INVENTION

In accordance with this invention, a method is provided for partially hydrolysing DFL to give at least one of 2′-FL or 3-FL. The invention thus provides a method for making a mixture containing at least one of 2′-FL and 3-FL, and optionally containing DFL, by subjecting DFL to partial hydrolysis. In carrying out this method, fucose (6-deoxy-L-galactose) and possibly lactose are produced as by-products. According to one aspect of the method, a partial hydrolysis of DFL mediated by a fucosidase provides a method for making one of 2′-FL or 3-FL, together with fucose. According to another aspect of the method, a partial hydrolysis of DFL mediated by an acid provides a method for making 2′-FL and/or 3-FL, together with fucose.

Also in accordance with this invention are provided:
a mixture consisting essentially of DFL, fucose and at least one of 2′-FL and 3-FL; and
a mixture consisting essentially of DFL, 2′-FL, 3-FL and fucose.

### BRIEF DESCRIPTION OF THE FIGURES

The invention will be described in further detail hereinafter with reference to the accompanying figures, in which:

FIG. 1 shows the hydrolysis of DFL with the 1,2-α-L-fucosidase from Xanthomonas manihotis ATCC# 49764 (bottom curve: t=0 h; upper curve: t=18 h); and

FIG. 2 shows the hydrolysis of DFL in 2′-FL and fucose using the α-1,3/4-fucosidase (Blon_{—}2336) from Bifidobacterium longum subsp. infantis atcc 15697 (chromatogram 1: t=0 h; chromatogram 2: t=18 h, [Enz. extract]=0.2 mg/ml; chromatogram 3: t=18 h, [Enz. extract]=2.0 mg/ml; chromatogram 4: t=18 h, [Enz. extract]=20 mg/ml).

### DETAILED DESCRIPTION OF THE INVENTION

By a partial hydrolysis of DFL, initiated by an acid or mediated by a fucosidase, a mixture containing at least one of 2′-FL and 3-FL (or both), and optionally containing DFL, can be obtained.

The first aspect of the invention is a method of making a mixture containing 2′-FL or 3-FL by subjecting DFL to partial hydrolysis mediated by a fucosidase. In carrying out this method, fucose is produced as a significant by-product and lactose can also be produced in small, not significant amounts, but preferably no lactose is produced.

For this partial hydrolysis, a fucosidase that can release one of the fucosyl residues from DFL is preferably used. In this regard, the partial hydrolysis of DFL with the fucosidase produces a selective hydrolysis. Depending on the activity of the fucosidase and/or its concentration and/or the length of time of the reaction, a mixture of DFL and at least one of 2′-FL and 3-FL can be obtained. This is particularly so when the enzyme treatment is carried out for a shorter time so as not to lead to total consumption of DFL.

One group of suitable fucosidases for this partial enzymatic hydrolysis belongs to the glycoside hydrolase family GH95 and are characterized by, and assigned to EC number 3.2.1.63. These 1,2-α-L-fucosidases cannot cleave the 3-O-fucosyl residue of DFL. As a result, they produce a mixture containing substantially no 2′-FL, i.e., no more than 2 w/w %, preferably no more than 1 w/w %, more preferably no more than 0.5 w/w % of 2′-FL, relative to 3-FL. (Previously, known 1,2-a-L-fucosidases have only been known to hydrolyse a 2-O-fucosyl residue from compounds having this fucosyl residue on a terminal galactose residue and not having another fucosyl residue on an adjacent sugar residue.) Preferably, the 1,2-α-L-fucosidase used in this method is one of those disclosed by the following references:

| Enzyme | Organism | Reference |
|---|---|---|
| α-1,2-L-fucosidase | Bifidobacterium bifidum | Katayama et al. |
| (AfcA) | JCM 1254 | J. Bacteriol. |
| | | 186, 4885 (2004) |
| α-1,2-L-fucosidase | Arabidopsis thaliana | Léonard et al. |
| (Fuc95A) | | Phytochemistry |
| | | 69, 1983 (2008) |
| α-1,2-L-fucosidase | Aspergillus nidulans | GenBank accession |
| (AN8149.2) | FGSC A4 | no.: EAA59171, |
| | | version EAA59171.1 |
| α-1,2-L-fucosidase | Lilium longiflorum | Ishimizu et al. J. Biochem. |
| (EBM II) | | 142, 721 (2007) |
| α-1,2-L-fucosidase | Bacillus cereus N521 | Miura et al. J. Biosci. |
| | | Bioeng. 99, 629 (2005) |
| α-1,2-L-fucosidase | Streptomyces sp. | Goso et al. Comp. |
| | OH11242 | Biochem. Physiol., B |
| | | 130, 375 (2001) |
| α-1,2-L-fucosidase | Xanthomonas manihotis | US 6,300,113 |
| | ATCC# 49764 | |

Another group of suitable fucosidases for this partial enzymatic hydrolysis belongs to the glycoside hydrolase family GH29 and are characterized by, and assigned to EC number 3.2.1.111. These 1,3/4-α-L-fucosidases cannot cleave the 2-O-fucosyl residue of DFL. As a result, they produce a mixture containing substantially no 3-FL, i.e., no more than 2 w/w %, preferably no more than 1 w/w %, more preferably no more than 0.5 w/w % 3-FL relative to 2′-FL. (Previously, known 1,3/4-α-L-fucosidases have only been known to hydrolyse a 3/4-O-fucosyl residue from compounds having this fucosyl residue on an intermediate N-acetyl-glucosamine or on a glucose residue and not having another fucosyl residue on an adjacent sugar residue.) Preferably, the 1,3/4-α-L-fucosidase used in this method is one of those disclosed by the following references:

| Enzyme | Organism | Reference |
|---|---|---|
| α-1,3/4-L-fucosidase | Bifidobacterium | Ashida et al. |
| (AfcB) | bifidum | Glycobiology 19, |
| | JCM 1254 | 1010 (2009) |
| α-1,3/4-L-fucosidase 1 | Arabidopsis | Zeleny et al. |
| (AtFUC1) | thaliana | Phytochemistry 64, |
| | | 641 (2006) |
| α-1,3/1,4-L-fucosidase | Bacteroides | GenBank accession no.: |
| (BT2192) | thetaiotaomicron | AAO77299, version |
| | VPI-5482 | AAO77299.1 |
| α-1,3/1,4-L-fucosidase | Bifidobacterium | Sela et al. Appl. |
| (Bion_2336) | longum | Environ. Microbiol. |
| | subsp. infantis | 78, 795 (2012) |
| | ATCC 15697 | |
| α-1,3/4-L-fucosidase | Streptomyces | Sano et al. J. Biol. |
| | sp. 142 | Chem. 267, |
| | | 1522 (1992) |
| α-1,3/4-L-fucosidase | Xanthomonas | US 6,300,113 |
| | manihotis | |
| | ATCC# 49764 | |

After the partial enzymatic hydrolysis, the fucosidase is denatured and centrifuged. The resulting solution, which contains fucose, is evaporated under reduced pressure. After lyophilisation, the dry residue is dissolved in water, and the mixture of 2′-FL or 3-FL with fucose and optionally with DFL is purified by biogel chromatography with water or by reverse phase chromatography. From the purified fractions, the mixture of 2′-FL or 3-FL with fucose and optionally with DFL can be isolated in solid form by crystallization, lyophilisation, precipitation or spray-drying, or in syrupy form. The above disclosed purification/separation method is suitable to separate 2′-FL or 3-FL from the mixture and to provide them in pure form.

The partial hydrolysis of this invention can also be carried out by subjecting DFL to partial acid hydrolysis. This involves gently treating DFL with an acid. The acid can be an organic or inorganic acid, preferably that having pKₐ less than 3-4. Preferred organic acids are formic acid, acetic acid, trichloroacetic acid, trifluoroacetic acid, oxalic acid, citric acid or sulfonic acids (like benzenesulfonic acid, p-toluenesulfonic acid, methanesulfonic acid, sulfonate type ion exchange resins), and preferred inorganic acids include mineral acids such as sulfuric acid, nitric acid, hydrochloric acid, perchloric acid, chloric acid, hydrobromic acid or hydroiodic acid, in the partial acid hydrolysis of this invention, one or both of the fucosyl residues of certain amounts of DFL are cleaved. Depending on the pH, the temperature and the length of time of the hydrolysis, a mixture of unhydrolysed DFL and partially hydrolysed DFL in the form of 2′-FL and optionally 3-FL in various proportions can be obtained. More precisely, the partial acid hydrolysis always provides more 2′-FL than 3-FL, thereby hydrolysing DFL in a selective manner. After the partial acid hydrolysis, the resulting mixture also contains a significant amount of fucose and some lactose.

Preferably, the partial acid hydrolysis of DFL produces a 2′-FL/3-FL ratio of more than 10. For this purpose, DFL is preferably treated with an organic acid, such as a carboxylic acid, e.g. formic acid, acetic acid, trichloroacetic acid, trifluoroacetic acid, oxalic acid or citric acid, for 2 or more hours but not more than 24 hours. Preferably, the hydrolysis is carried out at room temperature when the carboxylic acid is substituted with a halogen atom, or at more than 50° C., preferably at about 60-80° C., when the carboxylic acid is unsubstituted. Water and mixtures of water and organic protic or aprotic solvents which are stable under acidic conditions and fully or partially miscible with water, such as C₁-C₆ alcohols, acetone, THF, dioxane and MeCN, can be used as solvents In carrying out this partial acid hydrolysis, fucose is produced as a significant by-product and lactose can also be produced in small amounts, but preferably no significant amount of lactose is produced.The acid in the reaction mixture, containing the DFL, 2′-FL and optionally 3-FL can subsequently be neutralized in a conventional manner by the addition of an equivalent amount of a base, and the resulting salt can be removed by biogel filtration. Alternatively, a heterogeneous cationic ion exchange resin can be removed by simple filtration. From the resulting neutralized reaction mixture, the DFL, 2′-FL and optionally 3-FL can be separated in a conventional manner from the fucose and lactose by-products and then isolated in solid form by crystallization, lyophilisation, precipitation or spray-drying or in syrupy form.

In another preferred embodiment, the partial hydrolysis of DFL is carried out with an acid so as to achieve a mixture with a DFL/2′-FL ratio of from 1:3 to 1:8, preferably from 1:4 to 1:7, more preferably 1:5 to 1:6, particularly a ratio resembling that found in human mother's milk, that is a ratio of about 1:5.65, or a DFL/3-FL ratio of from 1:1 to 1:3, preferably from 1:1.5 to 1:2.5, particularly a ratio resembling that found in human mother's milk, that is a ratio of about 1:2, or a 3-FL/2′-FL ratio of from 1:1 to 1:5, preferably from 1:2 to 1:4, particularly a ratio resembling that found in human mother's milk, that is a ratio of about 1:2.8. I a most preferred embodiment the partial hydrolysis of DFL is carried out with an acid so as to achieve a mixture with a DFL/2′-FL/3-FL ratio resembling that found in human mother's milk, that is a ratio of about1:5.65:2.

Yet in another preferred embodiment, the partial hydrolysis of DFL is carried out with a fucosidase so as to achieve a mixture with a DFL/2′-FL ratio of from 1:3 to 1:8, preferably from 1:4 to 1:7, more preferably 1:5 to 1:6, particularly a ratio resembling that found in human mother's milk, that is a ratio of about 1:5.65, or a DFL/3-FL ratio of from 1:1 to 1:3, preferably from 1:1.5 to 1:2.5, particularly a ratio resembling that found in human mother's milk, that is a ratio of of about 1:2.

The method of this invention can readily provide the mixture consisting essentially of DFL, fucose and at least one of 2′-FL and 3-FL and preferably consisting essentially of DFL, fucose, 2′-FL and 3-FL. Preferably, the DFL/2′-FL/3-FL ratio in these mixtures resembles that found in human mother's milk, that is a ratio of about 1:5.65:2.

Both solid and liquid forms of the DFL/2′-FL/3-FL, DFL/2′-FL and DFL/3-FL mixtures of the present invention can be readily provided with a very high purity suitable for infant nutritional use, e.g. in infant formulas, infant cereals and clinical infant nutritional products. As a result, the solid and liquid forms of such mixtures are also suitable for general nutritional use for toddlers, children, adults and the elderly. The solid and liquid forms of such mixtures can also be used as food additives, dietary supplements, components of alcoholic and non-alcoholic beverages such as soft drinks, fruit juices, bottled water, wine and beer. The solid and liquid forms of such mixtures can also be used as therapeutic agents in broad therapeutic application areas, such as to prevent bacterial and viral infections, to avoid diarrhoea and to enhance immune system and brain development. The solid and liquid forms of such mixtures can also be used in veterinary applications, such as to fight against infectious diseases of domesticated animals.

The solid and liquid forms of the DFL/2′-FL/3-FL, DFL/2′-FL and DFL/3-FL mixtures of this invention are also suitable for pharmaceutical and/or therapeutic use. The solid and liquid forms of such mixtures, by themselves or in combination with other N-acetyllactosamine and/or lacto-N-biose and/or fucose and/or sialic acid containing human milk oligosaccharides, are particularly effective in the maturation of the immune system of neonatal infants and have preventive effect against secondary infections following viral infections such as influenza. The solid and liquid forms of such mixtures can be used as prebiotics to enhance the beneficial effects and efficiency of probiotics, such as Lactobacillus and Bifidobacterium species, in promoting the development of an early bifidogenic intestinal microbiota in infants, in reducing their risk of developing allergies and/or asthma and in preventing and treating pathogenic infections, such as diarrhoea, in infants.

The solid and liquid forms of the DFL/2′-FL/3-FL, DFL/2′-FL and DFL/3-FL mixtures of this invention can also be used as active ingredients in pharmaceutical compositions, together with one or more pharmaceutically acceptable carriers. Suitable carriers are described in the most recent edition of Remington's Pharmaceutical Sciences. Preferably, such pharmaceutical compositions also contain one or more conventional additives, adjuvants, excipients and diluents, such as water, gelatine, talc, sugars, starch, gum arabic, vegetable gums, vegetable oils, polyalkylene glycols, flavouring agents, preservatives, stabilizers, emulsifying agents, lubricants, colorants, fillers and wetting agents. The dosage form for administration of such pharmaceutical compositions can be, for example, conventional tablets, powders, granules, pills, suspensions, emulsions, infusions, capsules, syrups, injections, liquids, elixirs, extracts or tinctures. The solid and liquid forms of such mixtures can also be used for the preparation of such pharmaceutical compositions.

The solid and liquid forms of the DFL/2′-FL/3-FL, DFL/2′-FL and DFL/3-FL mixtures of this invention can further be used in food, drink or feed formulations. The amounts of such mixtures in these formulations can vary depending on whether the formulations are intended for use with normal, healthy infants, children, adults or the elderly or for use with individuals having specialized needs (e.g. suffering from metabolic disorders). Such nutritional formulations can also contain edible micronutrients, vitamins and minerals. Micronutrients can include, for example, edible oils, fats or fatty acids (such as coconut oil, soy-bean oil, monoglycerides, diglycerides, palm olein, sunflower oil, fish oil, linoleic acid or linolenic acid), carbohydrates (such as glucose, fructose, sucrose, maltodextrin, starch or hydrolysed cornstarch), and proteins from casein, soy-bean, whey or skim milk, or hydrolysates of these proteins, but protein from other sources (either intact or hydrolysed) can be used as well. Such nutritional formulations can also contain one or more of the following vitamins: A, B1, B2, B5, B6, B12, C, D, E, H, K, folic acid, inositol and nicotinic acid and one or more of the following minerals and trace elements: Ca, P, K, Na, Cl, Mg, Mn, Fe, Cu, Zn, Se, Cr or I. The solid and liquid forms of such mixtures can also be used for the preparation of such nutritional formulations

A preferred nutritional formulation containing a solid or liquid form of a DFL/2′-FL/3-FL, DFL/2′-FL or DFL/3-FL mixture of this invention is an infant formula, i.e., a foodstuff intended for particular nutritional use by infants during their first 4-6 months of life and satisfying by itself the nutritional requirements of infants. It may contain one or more probiotic Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g. palm olein, sunflower oil or safflower oil) and vitamins and minerals essential in a daily diet. The infant formula can contain the mixture of this invention in a total amount of 0.1-3.0 g/100 g formula.

Another preferred nutritional formulation is a food supplement containing a solid or liquid form of a DFL/2′-FL/3-FL, DFL/2′-FL or DFL/3-FL mixture of this invention. The food supplement can also contain one or more probiotics in an amount sufficient to achieve the desired effect in an individual, preferably in children and adults. The food supplement can also contain vitamins, minerals, trace elements and other micronutrients as well. The food supplement can be, for example, in the form of conventional tablets, capsules, pastilles or a liquid. The supplement can contain conventional additives, such as binders, coatings, emulsifiers, solubilising agents, encapsulating agents, film forming agents, adsorbents, carriers, fillers, dispersing agents, wetting agents, jellifying agents and gel forming agents. An especially preferred nutritional formulation is a digestive health functional food (e.g. in the form of tablets, capsules, powders or the like), used to enhance and preserve digestive health. (Such a nutritional formulation is also commonly called a dietary supplement or nutraceutical.)

### EXAMPLES

### Example 1

### Enzymatic Hydrolysis of DFL with 1,2-α-L-Fucosidase

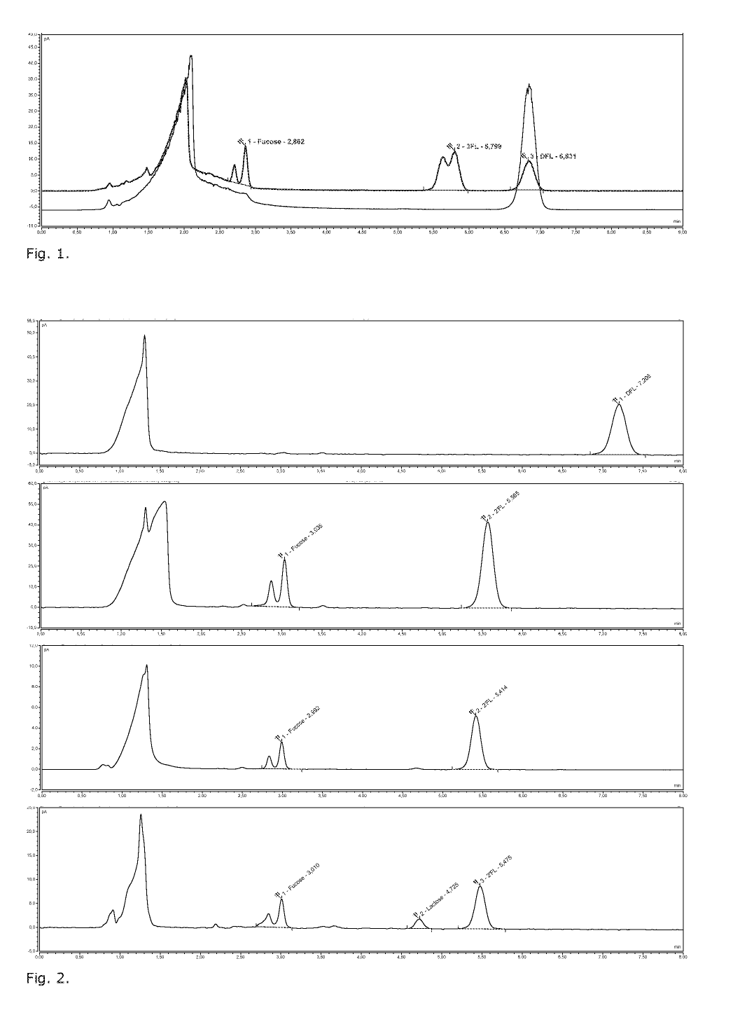

A solution of DFL (10 mM) was incubated with the 1,2-Ε-L-fucosidase from Xanthomonas manihotis ATCC# 49764 (500 U/ml) at 37° C. in an incubation buffer (50 mM sodium citrate, pH 6.0). Hydrolysis of DFL was followed by HPLC and new products were detected by CAD. As shown on FIG. 1 DFL was partially hydrolysed in 3-FL and fucose after 18 hours. No lactose could be detected.

### Example 2

### Enzymatic Hydrolysis of DFL with 1,3/4-α-L-Fucosidase

A solution of DFL (10 mM) was incubated with the 1,3/4-α-L-fucosidase Blon_{—}2336 from Bifidobacterium longum subsp. infantis atcc 15697 at 37° C. in an incubation buffer (100 mM potassium phosphate, pH 6.5). Different concentrations of enzyme extracts were used: 0.2 mg/ml, 2.0 mg/ml and 20 mg/ml as final concentration. Hydrolysis of DFL was followed by

HPLC and new products were detected by CAD. As shown on FIG. 2 DFL was fully hydrolysed in 2′-FL and fucose after 18 hours. Lactose could be detected only when the enzyme extract was used in a high concentration (20 mg/ml).

### Example 3

### Selective Hydrolysis of DFL with Acids

DFL (50 mg) was dissolved in water (0.5 ml) and treated with acid under the conditions indicated in the table below. Samples were analysed by HPLC using CAD detection.

| | | | relative area % | | | ratio |
|---|---|---|---|---|---|---|
| | | | DFL | 2′-FL + 3- FL | Fuc + Lac | 2′-FL: |
| acid | conditions | | left | formed | formed | 3-FL |
| acetic | 24 h | at rt | 100% | — | — | — |
| acid | +48 h | at 50° C. | 95.5% | 4% | 0.5%^{a} | 4.4 |
| (100 μl) | +4 days | at 50° C. | 89% | 8% | 3% | 9.1 |
| formic | 24 h | at rt | 100% | — | — | — |
| acid | +4 h | at 50° C. | 96% | 3% | 1%^{a} | 1.5 |
| (100 μl) | +48 h | at 50° C. | 71% | 17% | 12% | 15.7 |
| | +4 days | at 50° C. | 47.5% | 20% | 32.5% | only |
| | | | | | | 2′-FL |
| tosic | 24 h | at rt | 100% | — | — | — |
| acid | +4 h | at 50° C. | 96% | 2.5% | 1.5% | 5 |
| (10 mg) | +48 h | at 50° C. | 49% | 22% | 29% | 10.7 |
| | +4 days | at 50° C. | 22.5% | 14.5% | 63%^{b} | only |
| | | | | | | 2′-FL |
| sulfuric | 24 h | at rt | 94% | 4% | 2% | 3.6 |
| acid | +4 h | at 50° C. | 76.5% | 13.5% | 10% | 4.6 |
| (10 μl) | +48 h | at 50° C. | 13% | — | 87%^{c} | — |
| | +4 days | at 50° C. | — | — | 100%^{d} | — |
| Amberlite | 24 h | at rt | 100% | — | — | — |
| IR 120+ | +4 h | at 50° C. | 99% | 0.5% | 0.5%^{a} | 6 |
| | +48 h | at 50° C. | 85% | 8% | 7% | 10.1 |
| | +4 days | at 50° C. | 62% | 13% | 25% | 7.7 |

^{a}no Lac detected
^{b}includes also Glc and Gal (0.5%)
^{c}includes also Glc and Gal (2%)
^{d}includes also Glc and Gal (9%)

### Example 4

### Selective Hydrolysis of DFL with Acids

DFL (50 mg) was dissolved in 80% aq. acetic acid or 50% aq. TFA solution (250 μl) and stirred at the temperature indicated in the table below. Follow up of the reaction was monitored by HPLC (CAD detector).

| | | | relative area % | | | |
|---|---|---|---|---|---|---|
| | | | | 2′-FL + 3-FL | Fuc + Lac | ratio |
| acid | temperature | time | DFL left | formed | formed | 2′-FL:3-FL |
| 80% aq. acetic | 60° C. | 1 h | 100% | — | — | — |
| acid | | 4 h | 95.5% | 3.5% | 1% | 7.5 |
| | | 23 h | 80% | 13% | 7% | 14.9 |
| 80% aq. acetic | 80° C. | 1 h | 81% | 14% | 5% | 13.1 |
| acid | | 4 h | 48% | 26.5% | 25.5% | 17.9 |
| 50% aq. | rt | 1 h | 100% | — | — | — |
| trifluoroacetic | | 4 h | 89% | 8% | 3% | 38.5 |
| acid | | 23 h | 62.5% | 23.5% | 14% | 20.3 |
| | | 121 h | 15% | 12% | 73% | only 2′-FL present |

### Example 5

### Selective Hydrolysis of DFL with Acids

DFL (50 mg) was dissolved in a buffer solution (250 μl) and stirred at the temperature indicated in the table below. Follow up of the reaction was monitored by HPLC (CAD detector). Buffers were made according to “Electrolyte solutions” Robinson, R. A., and Stokes, R. H., 2nd ed., rev. London, Butterworths,1968, and “Practical chemistry” J. Lambert and T. A. Muir, 3rd. Ed. Heineman, London, 1973.

| | | relative area % | | | |
|---|---|---|---|---|---|
| | | | 2′-FL + | Fuc + Lac | ratio |
| pH | conditions | DFL left | 3-FL formed | formed | 2′-FL:3-FL |
| 1 | 50° C. for 15 h | 79.5% | 12.5% | 8% | 5.6 |
| | 50° C. for 40 h | 51% | 19.5% | 29.5% | 4.4 |
| | 50° C. for 69 h | 89% | 8% | 3% | 4.7 |
| 1.5 | 50° C. for 15 h | 96.5% | 2.5% | 1% | 4 |
| | 50° C. for 40 h | 84% | 10% | 6% | 4.5 |
| | 50° C. for 69 h | 79% | 12% | 9% | 3.4 |
| 2 | 15 h at 50° C. | 100% | — | — | — |
| | +25 h at 80° C. | 24.5% | 15% | 60.5% | 14 |
| | +29 h at 80° C. | 11% | — | 89%^{a} | — |
| 3 | 15 h at 50° C. | 100% | — | — | — |
| | +25 h at 80° C. | 88% | 8% | 4% | 5.3 |
| | +29 h at 80° C. | 71% | 16.5% | 12.5% | 4.5 |
| | +115 h at 80° C. | 36.5% | 21.5% | 42%^{b} | 8.4 |
| 4 | 80° C. for 72 h | 90% | 7% | 3% | 5.8 |
| | 80° C. for 112 h | 85% | 9.5% | 5.5% | 5.8 |

^{a}includes also Glc and Gal (3.5%)
^{b}includes also Glc and Gal (0.5%)

1. A method to form a mixture containing at least one of 2′-FL and 3-FL characterized in that DFL (difucosyl-lactose) is subjected to partial hydrolysis.

2. The method according to claim 1, wherein the mixture contains DFL and at least one of 2′-FL and 3-FL.

3. The method according to claim 1, which is mediated by a fucosidase.

4. The method according to claim 3, wherein the fucosidase a 1,2-α-L-fucosidase and the mixture contains DFL, 3-FL and substantially no 2′-FL.

5. The method according to claim 3, wherein the fucosidase is a 1,3/4-α-L-fucosidase and the mixture contains DFL, 2′-FL and substantially no 3-FL.

6. The method according to claim 3, wherein all the DFL is converted to one of 2′-FL or 3-FL.

7. The method according to claim 6, wherein all the DFL is converted to 3-FL by treatment with a 1,2-α-L-fucosidase.

8. The method according to claim 6, wherein all the DFL is converted to 2′-FL by treatment with a 1,3/4-α-L-fucosidase.

9. The method according to claim 1, wherein the mixture also contains fucose.

10. A mixture consisting essentially of DFL (difucosyl-lactose), fucose and at least one of 2′-FL and 3-FL.

11. The mixture according to claim 10 as a pharmaceutical agent.

12. A method of using the mixture according to claim 10 for the preparation of a pharmaceutical composition.

13. A pharmaceutical composition comprising the mixture according claim 10, and one or more pharmaceutically acceptable carriers.

14. A nutritional formulation comprising the mixture according to claim 10.

15. The nutritional formulation according to claim 14, which is an infant formula.

16. The nutritional formulation according to claim 14, which is a food supplement.

17. The food supplement according to claim 16, which is a digestive health functional food.

18. A method of using the mixture according to claim 10 for the preparation of a nutritional formulation.
